(19) United States
(12) Patent Application Publication
(10) Pub. No.: US 2004/0133014 A1
(43) Pub. Date: Jul. 8, 2004

(54) 3-Sulfenylation of indole-2-carboxylates

(76) Inventors: Harriet Wall Hamilton, Chelsea, MI (US); Alexei P. Krasutsky, Zionsville, IN (US); Jessica Reed, Ann Arbor, MI (US); Kevin Schlosser, Ann Arbor, MI (US)

Correspondence Address: Heidi M. Berven, Warner-Lambert Company, LLC, 2800 Plymouth Road, Ann Arbor, MI, 48105, US

(21) Appl. No.: 10/631,268

(22) Filed: Jul. 31, 2003

Related U.S. Application Data
(60) Provisional application No. 60/400,092, filed on Jul. 31, 2002.

(51) Int. Cl. C07D 209/36

(52) U.S. Cl. 548/484

(57) ABSTRACT

A highly efficient one-pot procedure for 3-sulfenilation of indole 2-carboxylates is described. Treatment of thiols with N-chlorosuccinimide at −78° C. in CH₂Cl₂ affords sulfenyl chlorides in situ that readily react with indole 2-carboxylates to give 3-thioindoles in high yields. This new method is milder, produces less waste, and is compatible with a wide range of thiol and indole functionality.

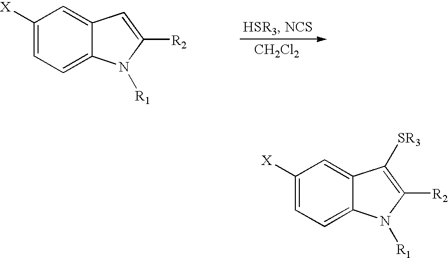

### CROSS REFERENCE TO RELATED APPLICATIONS

This application claims benefit of priority from U.S. Provisional Application No. 60/400,092, filed on Jul. 31, 2002.

### FIELD OF THE INVENTION

The invention is directed to a one-pot procedure for the 3-sulfenylation of indole-2-carboxylates.

### BACKGROUND OF THE INVENTION

Substituted indole-2-carboxylates, more specifically 3-thioindole-2-carboxylates, have been explored for their therapeutic worth in many fields including the treatment of HIV, obesity, as well as their use as endothelin antagonists and anti-allergy agents. The addition of sulfur at the 3-position of indole-2-carboxylates relies on the nucleophilicity of that center. Sulfur substitution at the 3-position using various forms of electrophilic sulfur including disulfides and sulfenyl chlorides has been reported. Many of these methods suffer from various shortcomings, however. For instance, while the oxidation of a thiol to a disulfide using sodium perborate typically proceeds cleanly in near quantitative yields, the subsequent reaction with indole produces an equivalent of thiol as an undesired by-product. Alternatively, formation of the sulfenyl chloride using sulfuryl chloride or chlorine often results in poor yields and is limited by the stability of the resulting sulfenyl chloride. The harsh conditions associated with chlorination reactions are also incompatible with certain functionalities. The formation of sulfenyl chlorides using N-chlorosuccinimide has also been reported. This milder method of chlorination effectively expands the scope of functional group compatibility, enabling the formation of thermally unstable aliphatic sulfenyl chlorides, including those with ester groups, but may require the isolation of the requisite sulfenyl chloride.

As a result, a need remains for an efficient technique for the introduction of sulfur at the 3-position of indole 2-carboxylates via a sulfenyl chloride that can be generated and used in situ.

### SUMMARY OF THE INVENTION

These and other needs are met by the present invention, which is directed to a one-step method for the sulfenylation of indole-2-carboxylates using in situ generated sulfenyl chlorides, comprising:

(a) mixing N-chlorosuccinimide and R₁SH in a liquid for sufficient temperatures and for a sufficient time to generate R₁SCl,

NCS+R₁SH→R₁SCl

wherein R₁ is (C₁-C₆)alkyl, (C₂-C₆)alkoxycarbonyl, (C₃-C₇)cycloalkyl, (C₃-C₇)heterocycloalkyl, (C₁-C₆)—S(O)ₘRₐ, —(C₁-C₆)—S(O)ₘNR_{b}R_{c}, (C₁-C₆)—NR_{b}R_{c}, or (C₁-C₆)—C(═O)—NR_{b}R_{c}, aryl, or heteroaryl, wherein (C₁-C₆)alkyl, (C₃-C₇)cycloalkyl, or (C₃-C₇)heterocycloalkyl is optionally partially unsaturated and (C₁-C₆)alkyl, (C₃-C₇)cycloalkyl, (C₃-C₇)heterocycloalkyl, aryl, or heteroaryl, is optionally substituted with aryl, aryl(C₁-C₆)alkoxy, aryloxy, arylcarbonyl, heteroaryl, heteroaryloxy, hydroxy, nitro, halo, cyano, (C₁-C₆)alkoxy, (C₁-C₆) alkanoyl, (C₁-C₆)alkoxycarbonyl, (C₁-C₆)alkanoyloxy, —S(O)ₘRₐ, —S(O)ₘNR_{b}R_{c}, NR_{b}R_{c}, or —C(═O) NR_{b}R_{c}, wherein m is 1 or 2 and a, b, and c are each independently H, (C₁-C₆)alkyl, (C₃-C₇)cycloalkyl, (C₃-C₆)heterocycloalkyl, or aryl;

(b) combining an indole-2-carboxylate 1 with the mixture containing the sulfenyl chloride generated in step (a) to provide the sulfenylated indole 2

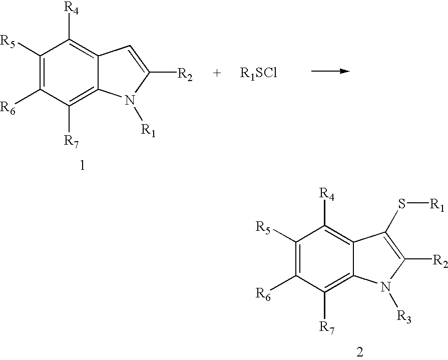

wherein R₁ is as provided in step (a);

R₂ is carboxy, tetrozolyl, (C₂-C₆)alkoxycarbonyl,

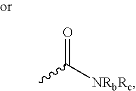

or —S(O)ₘRₐ, or —S(O)ₘNR_{b}R_{c}, NR_{b}R_{c}, or COR_{d}, optionally substituted with aryl, aryloxy, heteroaryl, heteroaryloxy, hydroxy, nitro, halo, or cyano, wherein R_{b} and R_{c} are each, independently H or (C₁-C₆)alkyl wherein m is 1 or 2 and a, b, and c are each independently H, (C₁-C₆)alkyl, (C₃-C₇)cycloalkyl, (C₃-C₆)heterocycloalkyl, or aryl; and

R₃ is H or (C₁-C₆)alkyl or (C₁-C₆)alkanoyl, optionally substituted with aryl, aryloxy, heteroaryl, heteroaryloxy, hydroxy, nitro, halo, or cyano;

R₄-R₇ are each independently H, halo, (C₁-C₆)alkyl, (C₁-C₆)alkoxy, cyano, (C₃-C₇)cycloalkyl, (C₃-C₇)heterocycloalkyl, (C₁-C₆)—S(O)ₘRₐ, —(C₁-C₆)—S(O)ₘNR_{b}R_{c}, (C₁-C₆)—NR_{b}R_{c}, or (C₁-C₆)—C(═O)—NR_{b}R_{c}, (C₁-C₆)—C(═O)R1, S(O)ₘRₐ, S(O)ₘNR_{b}R_{c}, NR_{b}R_{c}, C(═O)—NR_{b}R_{c}, C(═O)R_{d} aryl or heteroaryl, wherein (C₁-C₆)alkyl, (C₃-C₇)cycloalkyl, or (C₃-C₇)heterocycloalkyl is optionally partially unsaturated and (C₁-C₆)alkyl, (C₃-C₇)cycloalkyl, (C₃-C₇)heterocycloalkyl, aryl, or heteroaryl, is optionally substituted with aryl, aryloxy, heteroaryl, heteroaryloxy, hydroxy, nitro, halo, cyano, (C₁-C6)alkoxy, (C₁-C₆) alkanoyl, (C₁-C₆)alkoxycarbonyl, (C₁-C6)alkanoyloxy, —S(O)ₘRₐ, —S(O)ₘNR_{b}R_{c}, NR_{b}R_{c}, or —C(═O) NR_{b}R_{c}, C(═O)R₁ wherein m is 1 or 2 and a, b, and c are each independently H, (C₁-C₆)alkyl, (C₃-C₇)cycloalkyl, (C₃-C₆) heterocycloalkyl, heteroaryl or aryl, provided that not all of R₄-R₇ are H; and

(c) mixing the mixture generated in step b for sufficient temperature and for sufficient time to generate the sulfide.

The invention also provides a one-step method for the sulfenylation of indole-2-carboxylates using in situ generated sulfenyl chlorides, comprising:

(a) mixing N-chlorosuccinimide with compound 3 in a liquid for sufficient temperatures and for a sufficient time to generate compound 4,

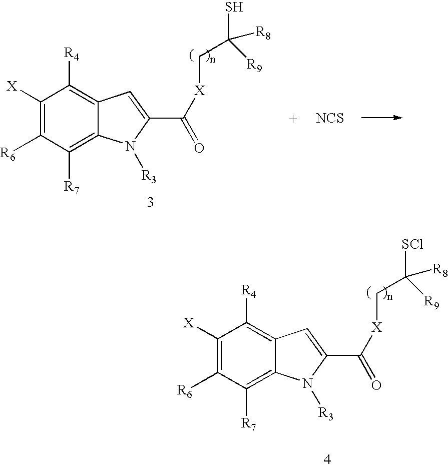

wherein R₃ is H or (C₁-C₆)alkyl or (C₁-C₆)alkanoyl, optionally substituted with aryl, aryloxy, heteroaryl, heteroaryloxy, hydroxy, nitro, halo, or cyano;

R₄- R6 and R₇ are independently H, halo, (C₁-C₆)alkyl, (C₁-C₆)alkoxy, Cyano, (C₃-C₇)cycloalkyl, (C₃-C₇)heterocycloalkyl, (C1-C6)—S(O)ₘRₐ, —(C1-C6)—S(O)ₘNR_{b}R_{c}, (C1-C6)—NR_{b}R_{c}, or (C1-C6)—C(═O)—NR_{b}R_{c}, (C1-C6)—C(═O)R1, S(O)ₘRₐ, S(O)ₘNR_{b}R_{c}, NR_{b}R_{c}, C(═O)—NR_{b}R_{c}, C(═O) R₁ aryl or heteroaryl, wherein (C₁-C₆)alkyl, (C₃-C₇)cycloalkyl, or (C₃-C₇)heterocycloalkyl is optionally partially unsaturated and (C₁-C₆)alkyl, (C₃-C₇)cycloalkyl, (C₃-C₇)heterocycloalkyl, aryl, or heteroaryl, is optionally substituted with aryl, aryloxy, heteroaryl, heteroaryloxy, hydroxy, nitro, halo, cyano, (C₁-C₆)alkoxy, (C₁-C₆) alkanoyl, (C₁-C₆)alkoxycarbonyl, (C₁-C₆)alkanoyloxy, —S(O)ₘRₐ, —S(O)ₘNR_{b}R_{c}, NR_{b}R_{c}, or —C(═O) NR_{b}R_{c}, C(═O)R1 wherein m is 1 or 2 and a, b, and c are each independently H, (C₁-C₆)alkyl, (C₃-C₇)cycloalkyl, (C₃-C₆) heterocycloalkyl, heteroaryl or aryl, provided that not all of R₄-R₇ are H;

R₈ and R₉ are independently H or (C₁-C₆)alkyl optionally substituted with aryl, aryloxy, heteroaryl, heteroaryloxy, hydroxy, nitro, halo, or cyano;

n is 0-4; and

X is CR₇R₈, O, or NR_{b}, wherein R_{b} is H, acyl, or (C₁-C₆)alkyl, optionally substituted with aryl, aryloxy, heteroaryl, heteroaryloxy, hydroxy, nitro, halo, or cyano; and

(b) allowing the sulfenyl chloride 4 generated in step (a) to provide the sulfenylated indole 5.

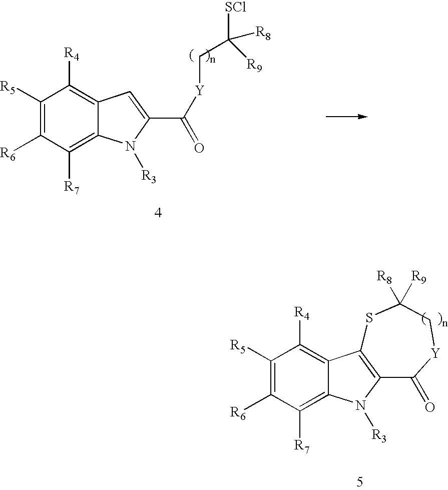

The advantages of this procedure include milder conditions than those associated with the use of corrosive chlorine or sulfuryl chloride, as well as fast reaction times, easy workup, and improved yields. The in situ formation method using NCS also enhances the scope of the reaction, previously limited by the stability and ease of isolation of the sulfenyl chlorides. The method also avoids the formation of one equivalent of wasted thiol that occurs when a disulfide is used as the electrophilic sulfur source.

In addition, the invention process provides a convenient approach to compounds that are useful as endothelin antagonists, as well as for HIV or obesity treatment.

### DETAILED DESCRIPTION OF THE INVENTION

The following definitions are used, unless otherwise described: halo is fluoro, chloro, bromo, or iodo. Alkyl, alkoxy, etc. denote both straight and branched groups; but reference to an individual radical such as “propyl” embraces only the straight chain radical, a branched chain isomer such as “isopropyl” being specifically referred to. When alkyl can be partially unsaturated, the alkyl chain may comprise one or more (e.g. 1, 2, 3, or 4) double or triple bonds in the chain.

Aryl and aryloxy denote an optionally substituted phenyl or phenoxy radical or an ortho-fused bicyclic carbocyclic radical having about nine to ten ring atoms in which at least one ring is aromatic. Heteroaryl denotes a radical of a monocyclic aromatic ring containing five or six ring atoms consisting of carbon and 1, 2, 3, or 4 heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

Arylcarbonyl refers to an optionally substituted phenyl radical attached to a carbonyl (“C═O”) moiety.

Aryl(C₁-C₆)alkoxy refers to an optionally substituted phenyl radical attached to a (C₁-C₆) alkoxy fragment.

Heterocycloalkyl is a cyclic, bicyclic ring or bridged system having from 4-10 atoms, from one to four of which are selected from O, S, and N. Heterocycle includes non-aromatic groups such as morpholino and pyrrolidino. Preferred heterocycles are 5- or 6-membered mono-cyclic aromatic rings having 1 or 2 heteroatoms. Heterocycle also includes bicyclic rings such as benzofuran, isothiazolone, indole, and the like. Heterocycle also includes bridged ring systems. Typical groups represented by the term include the following, wherein the hyphen indicates the point of attachment. The groups above and below are optionally substituted on the peripheral nitrogens by alkyl groups as defined above or by nitrogen protecting groups as described by Green (referenced above). Other typically preferred groups include pyrimidine, pyridazine, pyrazine, oxazole, pyrazole, thiazole, and the like. Most preferred are: piperazine, pyrrolidine, morpholine, thiomorpholine, thiazole, oxazole, isoxazole, piperidine, and azetidine.

The alkyl, cycloalkyl, aryl, aryloxy, heteroaryl, and heterocycloalkyl groups can be substituted with one or more groups selected from aryl, aryloxy, heteroaryl, heteroaryloxy, hydroxy, nitro, halo, or cyano.

The compounds of the present invention are generally named according to the IUPAC or CAS nomenclature system. Abbreviations which are well known to one of ordinary skill in the art may be used (e.g., “Ph” for phenyl, “Me” for methyl, “Et” for ethyl, “h” for hour or hours and “rt” for room temperature).

It will be appreciated by those skilled in the art that compounds of the invention having a chiral center may exist in and be isolated in optically-active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

The carbon atom content of various hydrocarbon-containing moieties is indicated by a prefix designating the minimum and maximum number of carbon atoms in the moiety, i.e., the prefix Cᵢ-Cⱼ indicates a moiety of the integer “i” to the integer “j” carbon atoms, inclusive. Thus, for example, (C₁-C₆)alkyl refers to alkyl of one to six carbon atoms, inclusive.

Specific and preferred values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkanoyl can be acetyl, propanoyl, butanoyl, pentanoyl, 4-methylpentanoyl, or hexanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide); heterocycloalkyl includes, tetrahydrofuranyl, pyrrolidinyl, piperidinyl, and the like.

Both intermolecular and intramolecular variants of the sulfenylation reaction are encompassed by the scope of the instant application.

1. Intermolecular Sulfenylation Reaction

Scheme 1 depicts the intermolecular variant of the sulfenylation method of the instant invention. In the first step of the reaction, the sulfenyl chloride is generated in situ by combining NCS with a thiol. In the second step of the reaction, an indole is combined with the in situ generated sulfenyl chloride to provide the sulfenylated indole product.

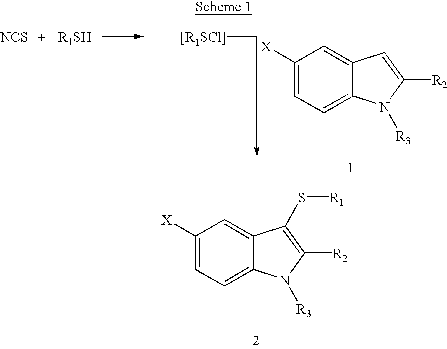

A. Thiol

A broad range of thiols may be used in the method of the present invention, including thiols wherein R₁ (C₁-C₆)alkyl, (C₂-C₆)alkoxycarbonyl, (C₃-C₇)cycloalkyl, (C₃-C₇)heterocycloalkyl, aryl, or heteroaryl, wherein (C₁-C₆)alkyl, (C₃-C₇)cycloalkyl, or (C₃-C₇)heterocycloalkyl, or aryl is optionally partially unsaturated and (C₁-C₆)alkyl, (C₃-C₇)cycloalkyl, (C₃-C₇)heterocycloalkyl, aryl, or heteroaryl, is optionally substituted with aryl, aryloxy, heteroaryl, heteroaryloxy, hydroxy, nitro, halo, cyano, (C₁-C₆)alkoxy, (C₁-C₆) alkanoyl, (C₁-C₆)alkoxycarbonyl, (C₁-C₆)alkanoyloxy, NR_{b}R_{c}, or —C(═O) NR_{b}R_{c}, and b, and c are each independently H, (C₁-C₆)alkyl, (C₃-C₇)cycloalkyl, (C₃-C₆)heterocycloalkyl, or aryl.

One group of thiols that may be used in the method of the present invention include thiols wherein R₁ in R₁SH is (C₁-C₆)alkyl or aryl, wherein (C₁-C₆)alkyl or aryl is optionally is optionally substituted with aryl, halo, cyano, (C₁-C₆)alkoxy, (C₁-C₆) alkanoyl, (C₁-C₆)alkoxycarbonyl, (C₁-C₆)alkanoyloxy, NR_{b}R_{c}, or —C(═O) NR_{b}R_{c}, and b, and c are each independently H, (C₁-C₆)alkyl, (C₃-C₇)cycloalkyl, (C₃-C₆)heterocycloalkyl, or aryl.

Another group of thiols that may be used in the method of the present invention include thiols wherein R₁ in R₁SH is is (C₁-C₆)alkyl or aryl, wherein (C₁-C₆)alkyl or aryl is optionally is optionally substituted with halo, cyano, (C₁-C₆)alkoxy, (C₁-C₆) alkanoyl, (C₁-C₆)alkoxycarbonyl, (C₁-C₆)alkanoyloxy, NR_{b}R_{c}, or —C(═O) NR_{b}R_{c}, and b, and c are each independently H, (C₁-C₆)alkyl, (C₃-C₇)cycloalkyl, or aryl.

Still another group of thiols that may be used in the method of the present invention include thiols wherein R₁ in R₁SH is t-butyl, phenyl, 2-, 3-, and 4-methoxyphenyl, benzyl, 2-, 3-, and 4-bromophenyl, 3-chloropropyl, 2-carbomethoxy ethyl, and 2-aminoethyl, wherein the amine moiety is protected as the BOC-amine or the like.

B. Indole 2-Carboxylates

Indole 2-carboxylates envisioned for use in the method of the present invention include compounds such as 2, depicted below.

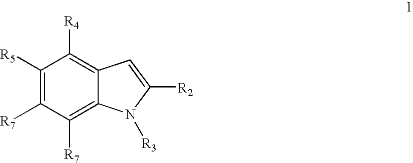

In compound 1, R₂ can be carboxy, tetrazolyl, alkoxycarbonyl,

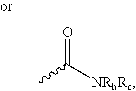

or —S(O)ₘRₐ, or —S(O)ₘNR_{b}R_{c}, NR_{b}R_{c}, or COR₁, optionally substituted with aryl, aryloxy, heteroaryl, heteroaryloxy, hydroxy, nitro, halo, or cyano, wherein R_{b} and R_{c} are each, independently H or (C₁-C6)alkyl wherein m is 1 or 2 and a, b, and c are each independently H, (C₁-C₆)alkyl, (C₃-C₇)cycloalkyl, (C₃-C₆)heterocycloalkyl, or aryl;

Specific values for R₂ include carboxy, (C₂-C₆)alkoxycarbonyl, or

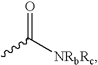

wherein R_{b} and R_{c} are each independently H or (C₁-C₆)alkyl; R₃ can be H or (C₁-C₆)alkyl. X can be H, halo or (C₁-C₆)alkoxy, and more specifically, carboxy, methoxycarbonyl, ethoxycarbony,

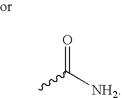

In compound 1, R₃ can be H or (C₁-C₆)alkyl or (C₁-C₆)alkanoyl, optionally substituted with aryl, aryloxy, heteroaryl, heteroaryloxy, hydroxy, nitro, halo, or cyano. A specific value for R₃ is CH₂CN.

In compound 1, R₄-R₇ independently can be H, halo, (C₁-C₆)alkyl, (C₁-C₆)alkoxy, Cyano, (C₃-C₇)cycloalkyl, (C₃-C₇)heterocycloalkyl, (C₁-C6)—S(O)ₘRₐ, —(C1-C6)—S(O)ₘNR_{b}R_{c}, (C1-C6)-NR_{b}R_{c}, or (C1-C6)—C(═O)—NR_{b}R_{c}, (C1-C6)—C(═O)R1, S(O)ₘRₐ, S(O)ₘNR_{b}R_{c}, NR_{b}R_{c}, C(═O)—NR_{b}R_{c}, C(═O) R₁ aryl or heteroaryl, wherein (C₁-C₆)alkyl, (C₃-C₇)cycloalkyl, or (C₃-C₇)heterocycloalkyl is optionally partially unsaturated and (C₁-C₆)alkyl, (C₃-C₇)cycloalkyl, (C₃-C₇)heterocycloalkyl, aryl, or heteroaryl, is optionally substituted with aryl, aryloxy, heteroaryl, heteroaryloxy, hydroxy, nitro, halo, cyano, (C₁-C₆)alkoxy, (C₁-C₆) alkanoyl, (C₁-C₆)alkoxycarbonyl, (C₁-C₆)alkanoyloxy, —S(O)ₘRₐ, —S(O)ₘNR_{b}R_{c}, NR_{b}R_{c}, or —C(═O) NR_{b}R_{c}, C(═O)R1 wherein m is 1 or 2 and a, b, and c are each independently H, (C₁-C₆)alkyl, (C₃-C₇)cycloalkyl, (C₃-C₆) heterocycloalkyl, heteroaryl or aryl.

C. Procedure and Stochiometry

As provided earlier, the invention process for sulfenylating indole-2-carboxylates embraces both intermolecular and intramolecular variants. In the intermolecular variant of the sulfenylation process of the present invention, a thiol is first contacted with NCS to generate the corresponding sulfenyl chloride. As used herein, “contacted” means that the reaction components are typically mixed in a liquid to form a homogeneous or heterogeneous mixture. The liquid employed in the sulfenylation process of the present invention is a polar aprotic solvent. Preferably, the polar aprotic solvent is selected from tetrahydrofuran, diethyl ether, acetonitrile, nitromethane, chloroform, methylene chloride, monochloro ethane, 1,1, or 1,2 dichloroethane, 1,1,1 or 1,1,2 tricholoroethane, or 1,1,1,2, or 1,1,2,2 tetrachloroethane. More preferred solvents include methylene chloride or chloroform. Mixtures of solvents can also be used.

To generate the sulfenyl chloride from the thiol, about 1 equivalent of NCS is used for each equivalent of thiol, although a slight excess (e.g., 1.01 to 1.2 equivalents) of NCS may be used to drive the chlorination reaction to completion.

The NCS and thiol in the liquid must be mixed at a sufficient concentration to ensure conversion of the thiol to the sulfenyl chloride. Thus, concentrations of NCS and thiol are typically in the range of about 0.05 to about 0.3 M for each respectively. More preferably, concentrations of NCS and thiol are typically in the range of about 0.1 to about 0.25 M each respectively. Concentrations of NCS and thiol are typically in the range of about 0.15 to about 0.2 M for each respectively.

The NCS and thiol in the liquid must be mixed for sufficient time to ensure conversion of the thiol to the sulfenyl chloride. Thus, reaction times are typically in the range of 5 minutes to an hour. More preferably, reaction times are typically in the range of 10 minutes to 30 minutes. More preferably, reaction times are typically in the range of 12 minutes to 20 minutes.

The NCS and thiol are mixed in the liquid at temperatures that are low enough to minimize or prevent undesired side reactions or NCS or sulfenyl chloride decomposition. Thus, the temperature of the mixture is typically in the range of −90 to −25° C. More preferably the temperature is in range of −80 to −20° C. More preferably the temperature is in the range of −79 to −70° C.

A solution of the indole-2-carboxylate in a solvent is then combined with the sulfenyl chloride generated during the first step of the invention method. Typically, the indole is added as a solution in a polar aprotic solvent such as methylene chloride, although other solvents such as diethylether, tetrahydrofuran, chloroform, or mixtures thereof, may be used. The solvent is used in an amount sufficient to produce a homogeneous mixture of the indole in the solvent. Typical concentrations of the indole in the solvent are thus in the range of about 0.1 to about 1.0 M. More preferably, concentrations are in the range of about 0.2 to about 0.9 M. More preferably, concentrations are in the range of about 0.3 to about 0.7 M The mixture of the indole in the solvent is added to the chilled mixture of the sulfenyl chloride at a rate sufficient to maintain the reaction temperature at below −70° C. The completion of the addition step culminates in the formation of a mixture containing sulfenyl chloride and indole. Typically, an excess of sulfenyl chloride is used based on the equivalents of indole used. Thus, about 1.01 to about 1.5 equivalents of sulfenyl chloride are used for each equivalent of indole used. More preferably, about 1.05 to about 1.3 equivalents of sulfenyl chloride are used for each equivalent of indole used. More preferably, about 1.09 to about 1.25 equivalents of sulfenyl chloride are used for each equivalent of indole used.

The mixture containing the sulfenyl chloride and indole is maintained at a temperature between −79 to −70° C. for up to about 15 to 60 minutes and then is allowed to warm to about 0° C. over the course of about 1 to 2 hours, although longer times may be necessary. Removal of the solvent by evaporation provides the crude sulfenylateted indole as a solid residue. The residue is then suspended in water and filtered. The sulfenylated indole product is collected as a solid, which may be further purified by recrystallization, in 40-100 percent yields generally.

In a typical procedure, the sulfenyl chloride of the desired thiol is formed in situ using N-chlorosuccinimide at −78° C. The indole is added after 15 minutes and the reaction is warmed to 0° C. over one hour. The solvent is evaporated and the residue suspended in water. Filtration of the mixture yields the desired product in high purity.

The sulfenyl chlorides prepared by the invention method are readily used in the direct functionalization of indoles. As Table 1 below indicates, the scope of this invention process possesses greater flexibility than other reported methods because the indole nitrogen does not require protection.

TABLE 1
Sulfenylations of indole-2-carboxylates

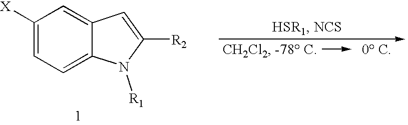

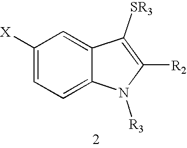

| Entry | X | R1 | R2 | HS-R3 | Yield |
|---|---|---|---|---|---|
| 1 | OMe | H | CO₂Me | 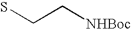 | 97 |
| 2 | OMe | Me | CO₂Me | 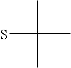 | 0 |
| 3 | OMe | Me | CO₂Me | 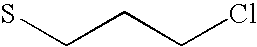 | 86 |
| 4 | OMe | H | CO₂Me | 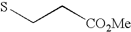 | 94 |
| 5 | OMe | Me | CO₂Me | 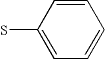 | 99 |
| 6 | OMe | Me | CONH₂ | 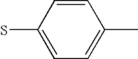 | 96 |
| 7 | OMe | Me | CONH₂ | 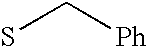 | 91 |
| 8 | OMe | Me | CONH₂ | 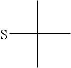 | 0 |
| 9 | H | H | CO₂Et | 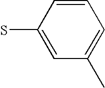 | 81* |
| 10 | H | H | CO₂Et | 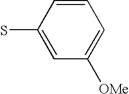 | 76* |
| 11 | H | H | CO₂Et | 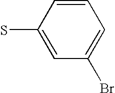 | 64* |
| 12 | F | H | CO₂Et | 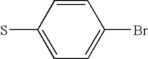 | 51* |
| 13 | F | H | CO2Et | 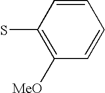 | 48* |

*Recrystallized yield

Table 1 also indicates that there is not a significant difference between yields in reactions employing protected versus unprotected indole cores. Moreover, a variety of thiols may be used, with the exception of tert-butyl thiol, which provides no reaction. Also, substitution in the indole does not appear to impede the sulfenylation reaction. However, the sulfenylation method has steric restrictions. For example, the reaction does not work for t-butyl thiol (entries 3 and 9 in Table 1).

2. Intramolecular Sulfenylation Reaction

Scheme 2 depicts the intramolecular variant of the sulfenylation method of the instant invention. The requisite thiol 2 is first prepared from the corresponding indole carboxylic acid using standard methodology. The sulfenyl chloride is next generated in situ, and then undergoes cyclization to provide the sulfenylated product 4.

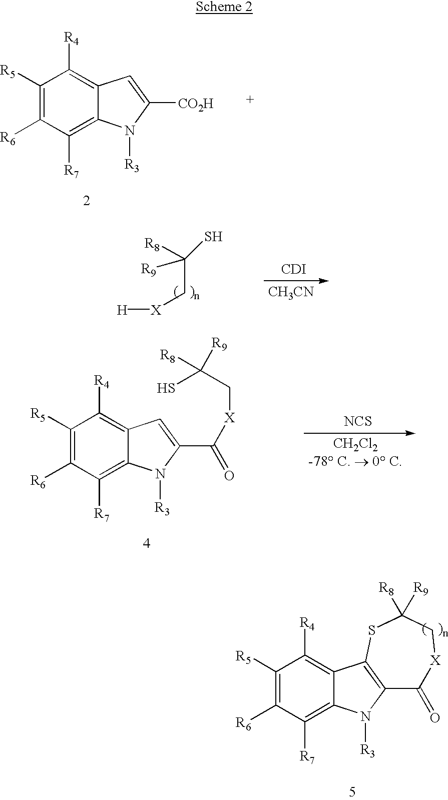

A. Thiol-Substituted Indole

A broad range of thiol-substituted indoles 4 may be used in the intramolecular variant of the present invention, including thiol substituted indoles wherein R₃-R₆ and X have any of the meanings provided above.

In addition, R₈ and R₉ independently in the thiol-substituted indole 4 can be H or (C₁-C₆)alkyl optionally substituted with aryl, aryloxy, heteroaryl, heteroaryloxy, hydroxy, nitro, halo, or cyano. Specific values for R₈ and R₉ include methyl, benzyl, isopropyl, and butyl and isobutyl.

Finally, X in the thiol-substituted indole 4 can be CR₇R₈, O, or NR_{b}, wherein R_{b} is H or (C₁-C₆)alkyl, optionally substituted with aryl, aryloxy, heteroaryl, heteroaryloxy, hydroxy, nitro, halo, or cyano.

A group of thiol-substituted indoles for use in the method of the instant invention includes compounds wherein one of R₄-R₇ is halo or alkoxy and the others are independantly are H or (C₁-C₆)alkyl, optionally substituted with aryl, aryloxy, heteroaryl, heteroaryloxy, hydroxy, nitro, halo, or cyano; R₇ and R₈ are independently H or methyl; n is 1, 2, or 3; X is H, halo, or methoxy; and Y us O or NR_{b}, wherein R_{b} is H, methyl, or acyl.

B Procedure and Stochiometry

As in the intermolecular variant of the sulfenylation process, in the intramolecular variant of the sulfenylation process, the thiol-substituted indole carboxylate is first contacted with NCS to generate the corresponding indole sulfenyl chloride. As used herein, “contacted” means that the reaction components are typically mixed in a liquid to form a homogeneous or heterogeneous mixture. The liquid employed in the sulfenylation process of the present invention is a polar aprotic solvent. Preferably, the polar aprotic solvent is selected from tetrahydrofuran, acetonitrile, nitromethane, chloroform, methylene chloride, monochloro ethane, 1,1, or 1,2 dichloroethane, 1,1,1 or 1,1,2 tricholoroethane, or 1,1,1,2, or 1,1,2,2 tetrachloroethane. More preferred solvents include methylene chloride or chloroform. Mixtures of solvents can also be used.

To generate the sulfenyl chloride from the thiol-substituted indole, about 1 equivalent of NCS is used for each equivalent of thiol-substituted indole, although a slight excess (e.g., 1.01 to 1.2 equivalents) of NCS may be used to drive the chlorination reaction to completion.

The NCS and thiol-substituted indole in the liquid must be mixed at a sufficient concentration to ensure conversion of the thiol to the sulfenyl chloride. Thus, concentrations of NCS and thiol are typically in the range of about 0.05 to about 0.3 M each respectively. More preferably, concentrations of NCS and thiol are typically in the range of about 0.1 to about 0.25 M each respectively. Concentrations of NCS and thiol are typically in the range of about 0.15 to about 0.2 M each respectively.

The NCS and thiol-substituted indole in the liquid must be mixed for sufficient time to ensure conversion of the thiol to the sulfenyl chloride. Thus, reaction times are typically in the range of 5 minutes to an hour. More preferably, reaction times are typically in the range of 10 minutes to 30 minutes. More preferably, reaction times are typically in the range of 12 minutes to 20 minutes.

The NCS and thiol-substituted indole are mixed in the liquid at temperatures that are low enough to minimize or prevent undesired side reactions or NCS sulfenyl chloride decomposition. Thus, the temperature of the mixture is typically in the range of −90 to −25° C. More preferably the temperature is in range of −80 to −20° C. More preferably the temperature is in the range of −79 to −70° C.

The indole sulfenyl chloride is maintained at a temperature between −79 to −70° C. for up to about 15 to 60 minutes and then is allowed to warm to about 0° C. over the course of about 1 to 2 hours, although longer times may be necessary. Removal of the solvent by evaporation provides the crude sulfenylateted indole as a solid residue. The residue is then suspended in water and filtered. The cyclized sulfenylated indole product is collected as a solid, which may be further purified by recrystallization.

A particular variant of the intramolecular method is depicted in Scheme 3.

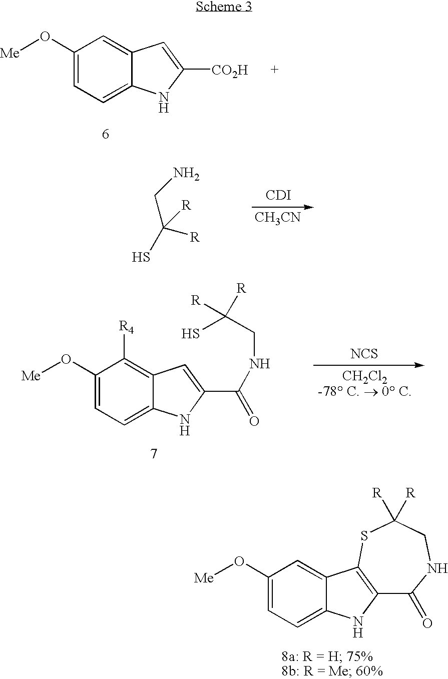

Thus, thioamide 7 was prepared from the corresponding indole-2-carboxylic acid 6 and 2-amino-thioethane via CDI amidation conditions. Reaction of 7 with NCS leads to cyclization and formation of previously unavailable thioazepines 8. The intramolecular reaction proceeds even for the sterically hindered gem-dimethyl substrate. A slight decrease in the yield of this reaction can be explained by chlorination of the 3-position of the indole as a side reaction.

3. Preparation of an Endothelin Antagonist Using the Invention Process

The invention process is easily adaptable to the synthesis of an array of biologically active molecules, for instance, compounds which are endothelin antagonists, or are useful in HIV or obesity treatment. For example, 1-Benzyl-3-(3-methoxy-phenylsulfanyl)-1H-indole-2-carboxylic acid 12 is an endothelin antagonist, as disclosed in U.S. Pat. No. 5,482,960. The compound can be prepared as provided in Scheme 4. Thus, indole-2-carboxylic acid methyl or ethyl ester is sulfenylated according to the invention process to provide 3-(3-Methoxy-phenylsulfanyl)-1H-indole-2-carboxylic acid 10. N-benzylation of compound 10 according to the procedure disclosed in U.S. Pat. No. 5,482,960 can give rise to compound 11, which may be hydrolyzed according to U.S. Pat. No. 5,482,960 using LiOH or any other procedure readily available to the skilled artisan to provide the target compound 12.

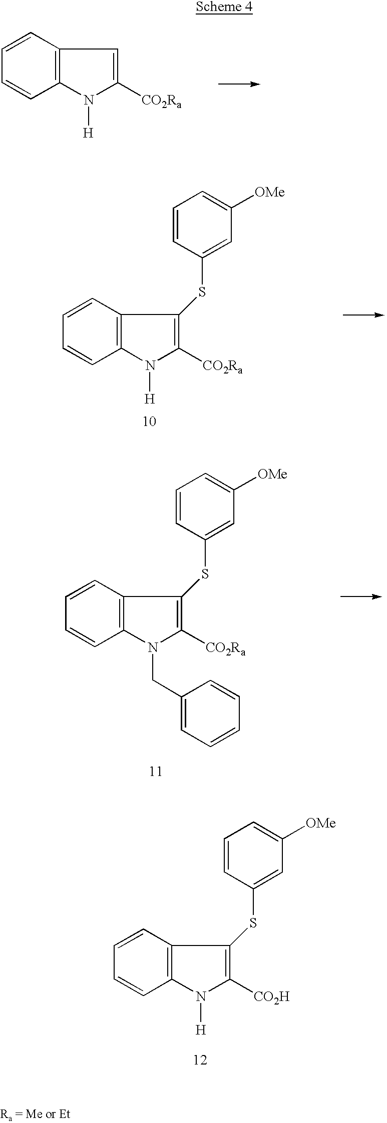

In conclusion, the invention provides a method for introduction of sulfur at the 3-position of indoles. This mild method is tolerant of a wide range of indole and thiol substrates that contain sensitive functionality. The high yielding reaction provides straightforward access to a wide array of potentially valuable targets.

The following examples are intended to illustrate various embodiments of the invention and are not intended to restrict the scope thereof.

### EXAMPLES

### Example 1

3-Methoxy-phenylsulfanyl-1H-indole-2-carboxylic acid methyl ester

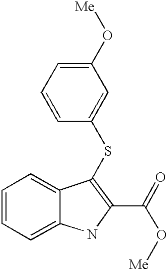

To a cooled solution of N-chlorosuccinimide (2.74 g, 20.6 mmol) in dichloromethane (125 mL) at −78° C., 3-methoxythiophenol (2.55 mL, 20.6 mmol) was added. The reaction was warmed to 0° C. over 15 minutes and a solution of indole-2-carboxylic acid methyl ester (3 g, 17.1 mmol) in dichloromethane (25 mL) was added. The reaction stirred at 0° C. for 1 hour, then concentrated under reduced pressure. The residue was suspended in H₂0 and stirred for 30 minutes. The solid was filtered and recrystallized from EtOAc/hexanes to yield the desired product (3.22 g, 60%). m.p.155-156° C. 500 MHz ¹H NMR (DMSO-d₆) δ 7.50 (d, 1H, J=7.6 Hz), 7.38 (d, 1H, J=7.6 Hz), 7.29 (t, 1H, J=7.1 Hz), 7.08 (m, 2H), 6.64 (d, 1H, J=7.6 Hz), 6.56 (m, 2H), 3.83 (s, 3H), 3.60 (s, 3H). MS nvz 314 (M+1). Anal. Calc'd for C₁₇H₁₅NO₃S C, 65.16; H, 4.82; N, 4.47; found: C,65.16; H,4.92: N, 4.40

### Example 2

4-Bromo-phenylsulfanyl-1H-indole-2-carboxylic acid methyl ester

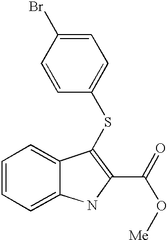

Prepared by the method described in Example 1 from 4-bromothiophenol to provide the desired ester (67%). 500 MHz ¹H NMR (DMSO-d₆): 12.47 (s, 1H), 7.51 (d, J=8.3 Hz, 1H), 7.39 (d, J=8.1 Hz, 1H), 7.36 (d, J=8.8 Hz, 2H), 7.30 (dd, J=8.3, 8.1 Hz, 1H), 7.09 (dd, J=8.3, 8.1 Hz, 1H), 6.96 (d, J=8.8 Hz, 2H), 3.82 (s, 3H). MS n/z 362, 364 (M+1).

### Example 3

3-m-Tolylsulfanyl-1H-indole-2-carboxylic acid methyl ester

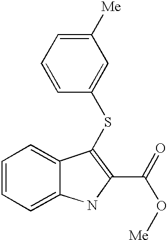

Prepared by the method described in Example 1 from 3-methylthiophenol to provide the desired ester (63% yield). 400 MHz ¹H NMR (DMSO-d₆) δ 7.50 (d, 1H, J=7.6 Hz), 7.38 (d, 1H, J=7.6 Hz), 7.29 (t, 1H, J=7.1 Hz), 7.08 (m, 2H), 6.64 (d, 1H, J=7.6 Hz), 6.56 (m, 2H), 3.83 (s, 3H), 3.60 (s, 3H). MS m/z 314 (M+1).

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

We claim:

1. A one-step method for the sulfenylation of 2-carboxyindoles comprising:
(a) mixing N-chlorosuccinimide and R₁SH in a liquid for sufficient temperatures and for a sufficient time to generate R₁SCl,
NCS+R₁SH→R₁SCl
wherein R₁ is (C₁-C₆)alkyl, (C₂-C₆)alkoxycarbonyl, (C₃-C₇)cycloalkyl, (C₃-C₇)heterocycloalkyl, (C₁-C₆)—S(O)ₘRₐ, —(C₁-C₆)—S(O)ₘNR_{b}R_{c}, (C₁-C₆)—NR_{b}R_{c}, or (C₁-C₆)—C(═O)—NR_{b}R_{c}, aryl, or heteroaryl, wherein (C₁-C₆)alkyl, (C₃-C₇)cycloalkyl, or (C₃-C₇)heterocycloalkyl is optionally partially unsaturated and (C₁-C₆)alkyl, (C₃-C₇)cycloalkyl, (C₃-C₇)heterocycloalkyl, aryl, or heteroaryl, is optionally substituted with aryl, aryl(C₁-C₆)alkoxy, aryloxy, arylcarbonyl, heteroaryl, heteroaryloxy, hydroxy, nitro, halo, cyano, (C₁-C₆)alkoxy, (C₁-C₆) alkanoyl, (C₁-C₆)alkoxycarbonyl, (C₁-C₆)alkanoyloxy, —S(O)ₘRₐ, —S(O)ₘNR_{b}R_{c}, NR_{b}R_{c}, or —C(═O) NR_{b}R_{c}, wherein m is 1 or 2 and a, b, and c are each independently H, (C₁-C₆)alkyl, (C₃-C₇)cycloalkyl, (C₃-C₆)heterocycloalkyl, or aryl;
(b) combining an indole-2-carboxylate 1 with the mixture containing the sulfenyl chloride generated in step (a) to provide the sulfenylated indole 2 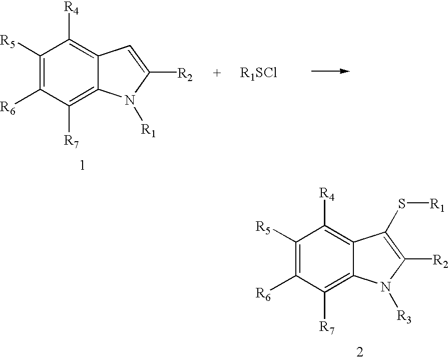
wherein R₁ is as provided in step (a);
R₂ is carboxy, tetrozolyl, (C₂-C₆)alkoxycarbonyl, 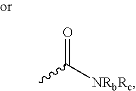
S(O)ₘRₐ, or —S(O),NR_{b}R_{c}, NR_{b}R_{c}, or COR_{d}, optionally substituted with aryl, aryloxy, heteroaryl, heteroaryloxy, hydroxy, nitro, halo, or cyano, wherein R_{b} and R_{c} are each, independently H or (C₁-C₆)alkyl wherein m is 1 or 2 and a, b, and c are each independently H, (C₁-C₆)alkyl, (C₃-C₇)cycloalkyl, (C₃-C₆)heterocycloalkyl, or aryl; and
R₃ is H or (C₁-C₆)alkyl or (C₁-C₆)alkanoyl, optionally substituted with aryl, aryloxy, heteroaryl, heteroaryloxy, hydroxy, nitro, halo, or cyano;
R₄-R₇ are each independently H, halo, (C₁-C₆)alkyl, (C₁-C₆)alkoxy, cyano, (C₃-C₇)cycloalkyl, (C₃-C₇)heterocycloalkyl, (C₁-C₆)—S(O)ₘRₐ, —(C₁-C₆)—S(O)ₘNR_{b}R_{c}, (C₁-C₆)—NR_{b}R_{c}, or (C₁-C₆)—C(═O)—NR_{b}R_{c}, (C₁-C6)—C(═O)R1, S(O)ₘRₐ, S(O)ₘNR_{b}R_{c}, NR_{b}R_{c}, C(═O)—NR_{b}R_{c}, C(═O)R_{d} aryl or heteroaryl, wherein (C₁-C₆)alkyl, (C₃-C₇)cycloalkyl, or (C₃-C₇)heterocycloalkyl is optionally partially unsaturated and (C₁-C₆)alkyl, (C₃-C₇)cycloalkyl, (C₃-C₇)heterocycloalkyl, aryl, or heteroaryl, is optionally substituted with aryl, aryloxy, heteroaryl, heteroaryloxy, hydroxy, nitro, halo, cyano, (C₁-C₆)alkoxy, (C₁-C₆) alkanoyl, (C₁-C₆)alkoxycarbonyl, (C₁-C₆)alkanoyloxy, —S(O)ₘRₐ, —S(O)ₘNR_{b}R_{c}, NR_{b}R_{c}, or —C(═O) NR_{b}R_{c}, C(═O)R₁ wherein m is 1 or 2 and a, b, and c are each independently H, (C₁-C₆)alkyl, (C₃-C₇)cycloalkyl, (C₃-C₆) heterocycloalkyl, heteroaryl or aryl, provided that not all of R₄-R₇ are H; and
(c) mixing the mixture generated in step b for sufficient temperature and for sufficient time to generate the sulfide.

2. The method of claim 1, wherein R₁ in R₁SH is (C₁-C₆)alkyl, (C₂-C₆)alkoxycarbonyl, (C₃-C₇)cycloalkyl, (C₃-C₇)heterocycloalkyl, aryl, or heteroaryl, wherein (C I-C₆)alkyl, (C₃-C₇)cycloalkyl, or (C₃-C₇)heterocycloalkyl, or aryl is optionally partially unsaturated and (C₁-C₆)alkyl, (C₃-C₇)cycloalkyl, (C₃-C₇)heterocycloalkyl, aryl, or heteroaryl, is optionally substituted with aryl, aryloxy, heteroaryl, heteroaryloxy, hydroxy, nitro, halo, cyano, (C₁-C₆)alkoxy, (C₁-C₆) alkanoyl, (C₁-C₆)alkoxycarbonyl, (C₁-C₆)alkanoyloxy, NR_{b}R_{c}, or —C(═O) NR_{b}R_{c}, and b, and c are each independently H, (C₁-C₆)alkyl, (C₃-C₇)cycloalkyl, (C₃-C₆)heterocycloalkyl, or aryl.

3. The method of claim 1, wherein R₁ in R₁SH is (C₁-C₆)alkyl or aryl, wherein (C₁-C₆)alkyl or aryl is optionally is optionally substituted with aryl, halo, cyano, (C₁-C₆)alkoxy, (C₁-C₆) alkanoyl, (C₁-C₆)alkoxycarbonyl, (C₁-C₆)alkanoyloxy, NR_{b}R_{c}, or —C(═O) NR_{b}R_{c}, and b, and c are each independently H, (C₁-C₆)alkyl, (C₃-C₇)cycloalkyl, (C₃-C₆)heterocycloalkyl, or aryl.

4. The method of claim 1, wherein R₁ in R₁SH is (C₁-C₆)alkyl or aryl, wherein (C₁-C₆)alkyl or aryl is optionally is optionally substituted with halo, cyano, (C₁-C₆)alkoxy, (C₁-C₆) alkanoyl, (C₁-C₆)alkoxycarbonyl, (C₁-C₆)alkanoyloxy, NR_{b}R_{c}, or —C(═O) NR_{b}R_{c}, and b, and c are each independently H, (C₁-C₆)alkyl, (C₃-C₇)cycloalkyl, (C₃-C₆)heterocycloalkyl, or aryl.

5. The method of claim 1, wherein R₁ in R₁SH is (C₁-C₆)alkyl or aryl, wherein (C₁-C₆)alkyl or aryl is optionally is optionally substituted with halo, cyano, (C₁-C₆)alkoxy, (C₁-C₆) alkanoyl, (C₁-C₆)alkoxycarbonyl, (C₁-C₆)alkanoyloxy, NR_{b}R_{c}, or —C(═O) NR_{b}R_{c}, and b, and c are each independently H, (C₁-C₆)alkyl, (C₃-C₇)cycloalkyl, or aryl.

6. The method of claim 1, wherein contacting in step (a) comprises mixing the NCS and thiol in a liquid to form a homogeneous or heterogeneous mixture.

7. The method of claim 1, wherein the liquid in step (a) is a polar aprotic solvent selected from tetrahydrofuran, diethyl ether, acetonitrile, nitromethane, chloroform, methylene chloride, monochloro ethane, 1,1, or 1,2 dichloroethane, 1,1,1 or 1,1,2 tricholoroethane, or 1,1,1,2, or 1,1,2,2 tetrachloroethane or combinations thereof.

8. The method of claim 1, wherein the liquid in step (a) is methylene chloride or chloroform or mixtures thereof.

9. The method of step (a) of claim 1, wherein about 1.01 to about 1.2 equivalent of NCS is used for each equivalent of thiol.

10. The method of step (a) of claim 1, wherein the concentrations of NCS and thiol are typically in the range of about 0.05 to about 0.3 M each respectively.

11. The method of step (a) of claim 1, wherein the concentration of NCS and thiol are typically in the range of about 0.1 to about 0.25 M each respectively.

12. The method of step (a) of claim 1, wherein concentrations of NCS and thiol are typically in the range of about 0.15 to about 0.2 M each respectively.

13. The method of step (a) of claim 1, wherein reaction times are in the range of about 10 minutes to about 30 minutes.

14. The method of step (a) of claim 1, wherein reaction times are in the range of 12 minutes to 20 minutes.

15. The method of step (a) of claim 1, wherein reaction times are 15 minutes.

16. The method of step (a) of claim 1, wherein the NCS and thiol are mixed in the liquid at temperatures in the range of about −90 to −25° C.

17. The method of step (a) of claim 1, wherein wherein the NCS and thiol are mixed in the liquid at temperatures in the range of about −79 to −70° C.

18. The method of step (b) of claim 1, wherein the indole-2-carboxylate in a solvent is added to the sulfenyl chloride generated during step (a) of claim 1.

19. The method of step (b) of claim 1, wherein the indole is added as a solution in a polar aprotic solvent as recited in claim 7.

20. The method of step (b) of claim 1, wherein the indole is added as a solution in a polar aprotic solvent is methylene chloride.

21. The method of step (b) of claim 1, wherein the concentration of the indole in the solvent is between about 0.1 to about 1.0 M.

22. The method of step (b) of claim 1, wherein the concentration of the indole in the solvent is between about 0.2 to about 0.9 M.

23. The method of step (b) of claim 1, wherein the concentration of the indole in the solvent is between about 0.3. to about 0.7 M.

24. The method of step (b) of claim 1, wherein the mixture of the indole in the solvent is added to the chilled mixture of the sulfenyl chloride at a rate sufficient to maintain the reaction temperature at below −70° C. The completion of the addition step culminates in the formation of a mixture containing sulfenyl chloride and indole.

25. The method of step (b) of claim 1, wherein about 1.01 to about 1.5 equivalents of sulfenyl chloride are used for each equivalent of indole used.

26. The method of step (b) of claim 1, wherein about 1.05 to about 1.3 equivalents of sulfenyl chloride are used for each equivalent of indole used.

27. The method of step (b) of claim 1, wherein about 1.09 to about 1.25 equivalents of sulfenyl chloride are used for each equivalent of indole used.

28. The method of step (b) of claim 1, wherein the mixture containing the sulfenyl chloride and indole is maintained at a temperature between about −79 to −70° C. for up to about 15 to 60 minutes and then is allowed to warm to about 0° C. over the course of about 1 to 2 hours.

29. The method of step (c) of claim 1, wherein about the solvent from step (b) of claim 1 is removed by evaporation.

30. A method for the intramolecular sulfenylation of 2-carboxyindoles comprising comprising:
(a) mixing N-chlorosuccinimide with compound 3 in a liquid for sufficient temperatures and for a sufficient time to generate compound 4, 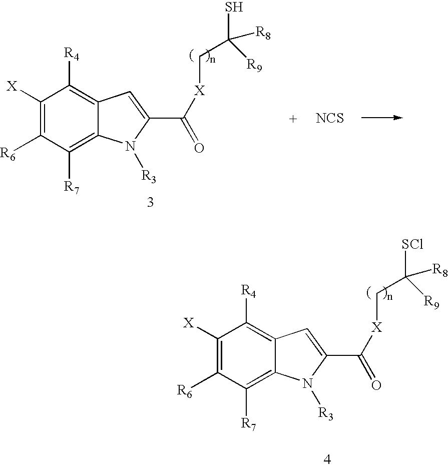
wherein R₃ is H or (C₁-C₆)alkyl or (C₁-C₆)alkanoyl, optionally substituted with aryl, aryloxy, heteroaryl, heteroaryloxy, hydroxy, nitro, halo, or cyano;
R₄- R₆ and R₇ are independently H, halo, (C₁-C₆)alkyl, (C₁-C₆)alkoxy, Cyano, (C₃-C₇)cycloalkyl, (C₃-C₇)heterocycloalkyl, (C₁-C₆)—S(O)ₘRₐ, —(C₁-C₆)—S(O)ₘNR_{b}R_{c}, (C₁-C6)—NR_{b}R_{c}, or (C₁-C₆)—C(═O)—NR_{b}R_{c}, (C₁-C6)—C(═O)R1, S(O)ₘRₐ, S(O)ₘNR_{b}R_{c}, NR_{b}R_{c}, C(═O)—NR_{b}R_{c}, C(═O) R₁ aryl or heteroaryl, wherein (C₁-C₆)alkyl, (C₃-C₇)cycloalkyl, or (C₃-C₇)heterocycloalkyl is optionally partially unsaturated and (C₁-C₆)alkyl, (C₃-C₇)cycloalkyl, (C₃-C₇)heterocycloalkyl, aryl, or heteroaryl, is optionally substituted with aryl, aryloxy, heteroaryl, heteroaryloxy, hydroxy, nitro, halo, cyano, (C₁-C₆)alkoxy, (C₁-C₆) alkanoyl, (C₁-C₆)alkoxycarbonyl, (C₁-C₆)alkanoyloxy, —S(O)ₘRₐ, —S(O)ₘNR_{b}R_{c}, NR_{b}R_{c}, or —C(═O) NR_{b}R_{c}, C(═O)R1 wherein m is 1 or 2 and a, b, and c are each independently H, (C₁-C₆)alkyl, (C₃-C₇)cycloalkyl, (C₃-C₆) heterocycloalkyl, heteroaryl or aryl, provided that not all of R₄-R₇ are H;
R₈and R₉are independently H or (C₁-C₆)alkyl optionally substituted with aryl, aryloxy, heteroaryl, heteroaryloxy, hydroxy, nitro, halo, or cyano;
n is 0-4; and
X is CR₇R₈, O, or NR_{b}, wherein R_{b} is H, acyl, or (C₁-C₆)alkyl, optionally substituted with aryl, aryloxy, heteroaryl, heteroaryloxy, hydroxy, nitro, halo, or cyano; and
(b) allowing the sulfenyl chloride 4 generated in step (a) to provide the sulfenylated indole 5.

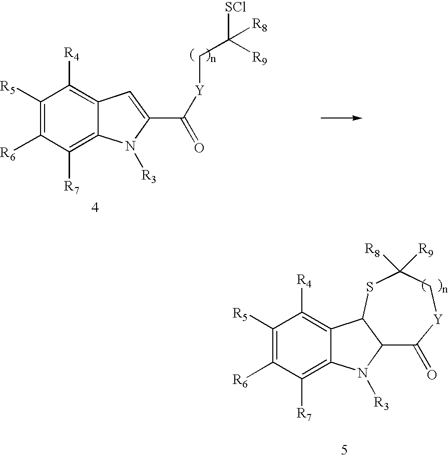

31. The method of claim 30, wherein R₃ in compounds 4-6 is H and (C₁-C₆)alkyl,;
R₄ and R₅ are independently H or (C₁-C₆)alkyl;
n is 1, 2, or 3; and
X is H, halo or (C₁-C₆)alkoxy.

32. The method of claim 31, wherein R₃ in compounds 4-6 is H or methyl;
R₄ and R₅ are independently H or methyl;
n is 1, 2, or 3; and
X is H, halo or methoxy.

33. The method of claim 30, wherein contacting in step (a) comprises mixing the NCS and thiol in a liquid to form a homogeneous or heterogeneous mixture.

34. The method of claim 30, wherein the liquid in step (a) is a polar aprotic solvent as recited in claim 7.

35. The method of claim 30, wherein the liquid in step (a) is methylene chloride or chloroform or mixtures thereof.

36. The method of step (a) of claim 30, wherein reaction times are in the range of about 10 minutes to about 30 minutes.

37. The method of step (a) of claim 30, wherein reaction times are in the range of 12 minutes to 20 minutes.

38. The method of step (a) of claim 30, wherein reaction times are 15 minutes.

39. The method of step (a) of claim 30, wherein the NCS and thiol-substituted indole are mixed in the liquid at temperatures in between about −90 to −25° C.

40. The method of step (a) of claim 30, wherein wherein the NCS and thiol-substituted indole are mixed in the liquid at temperatures in between about −79 to −70° C.

41. The method of step (a) of claim 30, wherein about 1.01 to about 1.2 equivalent of NCS is used for each equivalent of thiol-substituted indole.

42. The method of step (a) of claim 1, wherein the concentration of the thiol-substituted indole in the solvent is between about 0.1 to about 1.0 M.

43. The method of step (a) of claim 30, wherein the concentration of the thiol-substituted indole in the solvent is between about 0.2 to about 0.9 M.

44. The method of step (a) of claim 30, wherein the concentration of the thiol-substituted indole in the solvent is between about 0.3. to about 0.7 M.

45. The method of step (b) of claim 30, wherein the mixture of the thiol-substituted indole in the solvent is added to the chilled mixture of the sulfenyl chloride at a rate sufficient to maintain the reaction temperature at below −70° C. The completion of the addition step culminates in the formation of a mixture containing sulfenyl chloride and indole.

46. The method of step (b) of claim 30, wherein about 1.01 to about 1.5 equivalents of sulfenyl chloride are used for each equivalent of thiol-substituted indole used.

47. The method of step (b) of claim 30, wherein about 1.05 to about 1.3 equivalents of sulfenyl chloride are used for each equivalent of thiol-substituted indole used.

48. The method of step (b) of claim 30, wherein about 1.09 to about 1.25 equivalents of sulfenyl chloride are used for each equivalent of thiol-substituted indole used.

49. The method of step (b) of claim 30, wherein the mixture containing the sulfenyl chloride and thiol-substituted indole is maintained at a temperature between about −79 to −70° C. for up to about 15 to 60 minutes and then is allowed to warm to about 0° C. over the course of about 1 to 2 hours.

50. The method of step (c) of claim 30, wherein the solvent from step (b) of claim 30 is removed by evaporation.
